(19) United States
(12) Patent Application Publication
(10) Pub. No.: US 2013/0058872 A1
(43) Pub. Date: Mar. 7, 2013

(54) Bioluminescent Endoscopy Methods and Compounds

(71) Applicants: Philip T. Neff, Golden, CO (US); Bruce Bryan, Beverly Hills, CA (US); Randall B. Murphy, Glenmoore, PA (US)

(72) Inventors: Philip T. Neff, Golden, CO (US); Bruce Bryan, Beverly Hills, CA (US); Randall B. Murphy, Glenmoore, PA (US)

(21) Appl. No.: 13/610,242

(22) Filed: Sep. 11, 2012

Related U.S. Application Data
(63) Continuation-in-part of application No. 12/201,680, filed on Aug. 29, 2008.
(60) Provisional application No. 60/968,772, filed on Aug. 29, 2007.

(51) Int. Cl. A61K 49/00

(52) U.S. Cl. 424/9.6

(57) ABSTRACT

Bioluminescent endoscopy methods in which bioluminescent generating system components are combined ex vivo immediately prior to injection or infusion into a tissue area of interest to enable bioluminescent viewing of the tissue. The bioluminescent generating system components are combined by simultaneous injection into a Y tube or other in-line mixing chamber receiving fluid inputs from two or more fluid conduits and providing a fluid output to at least one fluid conduit. The mixed components which are the output of such multi lumen mixing system is injected into a hollow viscus organ or otherwise applied to organs and/or tissues of the body.

### CROSS-REFERENCE TO RELATED APPLICATIONS

The present application is a continuation-in-part of pending U.S. patent application Ser. No. 12/201,680, filed Aug. 29, 2008, which claims the benefit under 35 U.S.C. §119(e) of the U.S. Provisional Patent Application Ser. No. 60/968,772, filed Aug. 29, 2007, the contents of both of which are incorporated herein by reference.

### FIELD OF THE INVENTION

The present invention relates to methods and compositions for endoscopic diagnosis and surgical treatment in a living animal, bird, or man using bioluminescent systems.

### BACKGROUND OF THE INVENTION

In recent years surgical endoscopes capable of viewing a patient or test subject have been widely employed. Surgical endoscopes are commonly used to observe organs or other structures, and can be used for providing therapeutic treatments or surgical interventions by inserting treatment tools into a treatment tool channel provided therein. A typical example of this would be a rigid endoscope including a hard insertion section, which incorporates an image capturing device. With this rigid endoscope, a light guide cable and a scope cable are integrated within the rigid endoscope main body. Flexible endoscopes may also be used to observe anatomic structures. Many different types of electronic endoscopes using a solid state imaging device such as a charge coupled device (CCD) or complementary metal-oxide-semiconductor sensor (CMOS) as imaging means have also been considered and/or used.

However, certain anatomical structures, particularly viscus organs, are difficult to observe in conventional endoscopes under normal visible lighting conditions. For example, the common be duct is not infrequently damaged in laparoscopic resection of the gallbladder because of its size, location and visual perception limitations inherent in endoscopic surgical techniques (Way L W, Stewart L, Gantert W, Liu K, Lee C M, Whang K, Hunter J G. “Causes and prevention of laparoscopic bile duct injuries: analysis of 252 cases from a human factors and cognitive psychology perspective.” Annals of Surgery 2003 April; 237(4):460-9). Similarly, the ureter can be damaged in surgery, especially in pelvic operations. Although these injuries can be repaired, they may not be apparent until after the conclusion of the procedure. Commonly in the case of the bile duct, infusion of the duct structures with an X-ray contrast agent may be performed, and an X-ray photograph or fluoroscopic image is taken in the form of an intraoperative cholangiogram. This necessitates stopping the surgical procedure and requires bringing an X-ray machine into the operating theater. Furthermore, the operator and assistants must put on lead gowns making this procedure inconvenient and time consuming. For this reason, many surgeons may be resistant to this X-ray examination. Upon an extensive review on the role of intraoperative cholangiography in avoiding bile duct injury, the data suggest that the use and correct interpretation of IOC decreases the rate of common bile duct injury and that its broader use will improve patient safety (Massarweh N N, Rum D R. (2007). “Role of intraoperative cholangiography in avoiding bile duct injury.” Journal American College Surgery 204(4):656-64. However, selective intraoperative cholangiography as compared to routine intraoperative cholangiography (IOC) techniques in itself increases the risk of injury to the common bile duct (Flum D R, Dellinger E P, Cheadle A, Chan L, Koepsell T (2003) “Intraoperative choiangiography and risk of common bile duct injury during cholecystectomy.” Journal American Medical Association 289(13): 1639-44).

Experiments have been made using bioluminescent systems wherein luciferin and luciferase components have been separately injected through separate cannulas and/or applied to an organ and combined in situ. Such experiments have demonstrated the ability to illuminate organs, however, in some examples this approach has been found to be unsatisfactory as parts of an organ may not be illuminated if circulation and mixing is obstructed or prevented. Also, illumination levels can be inconsistent within the organ.

However, heretofore it has been considered not feasible to engage in ex vivo mixing of luciferin and luciferase components with subsequent injection or application to organs. Generally, the time period of effective illumination in bioluminescent systems is very short and the degree of illumination is at or below the limit of detectability of an endoscopic system As described in more detail herein, the present invention provides for improved methods of administering a bioluminescent compound or mixtures of bioluminescent compounds to enable bioluminescent endoscopy methods used to avoid such problems in surgery.

### SUMMARY OF THE INVENTION

The present invention relates to methods of ex vivo mixing of bioluminescent compounds immediately before delivery to a tissue area of interest to enable bioluminescent endoscopy methods, by which images may be obtained by the use of appropriate cameras and signal processing algorithms, if necessary, from tissues which contain a mixture of bioluminescent compounds which are injected, administered, or by any means whatsoever placed within said tissue. The present invention provides a method of using a bioluminescence system to illuminate internal body structures of a subject by combining a luciferase and a bioluminescence substrate and any required activator using a Y tube or other mufti lumen mixing system and injection of the mixture into a viscus organ or other administration to organs of the body, followed by visualizing and/or imaging the subject or one or more portions thereof.

In certain embodiments, the method is used for illumination of one or more components of a circulatory system, nervous system, body organ, or lymphatic system of the subject. In preferred embodiments, one or more of the components is a hollow viscus organ. such as blood vessels, or the colon, ureter, bladder, bile duct, or cystic duct.

In preferred embodiments of the method, the step of administering comprises injecting, perfusing, administering, superfusing, infiltrating, or otherwise applying the media to internal body structures to provide bioluminescent illumination to the body structures and the step of visualizing and/or imaging comprises obtaining one or more images of the structure with bioluminescent illumination with a camera system sensitive to the wavelength of emission of the bioluminescence system.

In some of the preferred embodiments, the one or more images are obtained in dim visible light conditions.

In other of the preferred embodiments, the one or more images are color or monochrome images.

In yet other preferred embodiments, the step of visualizing and/or imaging further comprises obtaining one or more images of the object with visible light illumination, the visible light comprising visible light in a wavelength range which does not include light in a wavelength range of light emitted by the bioluminescence system. In some of these embodiments, the images are obtained with a camera system sensitive to the wavelength of emission of the visible light illumination. In certain of these embodiments, the visible light illumination is a red light illumination. In certain preferred embodiments, the method further comprises comparing the image with bioluminescent illumination with said image with visible light illumination. In some of these embodiments, the method further comprises a step of switching between said image with bioluminescent illumination and said image with visible light illumination.

In certain embodiments of the method, the object comprises a component of an animal, avian or human circulatory system, nervous system, body organ, or lymphatic system. In some of these embodiments, the object comprises gallbladder, ureter, lung, heart, or intestine.

In some embodiments of the method, the images are obtained by an endoscopic examination of the object.

The invention also comprises a method of using a bioluminescence system post-operatively to illuminate a surgical tissue in order to inspect the surgical tissue comprising providing a luciferase and a bioluminescence substrate, combining in the presence of an activator the luciferase and the bioluminescence substrate to form a bioluminescent media, administering to the subject or one or more portions thereof the bioluminescent media and visualizing and/or imaging said subject or one or more portions thereof.

In preferred embodiments of the post-operative method, the step of administering comprises perfusing, administering, injecting, superfusing, infiltrating, or otherwise applying the media to internal body structures to provide bioluminescent illumination to the body structures and the step of visualizing and/or imaging comprises obtaining one or more images of the structure with bioluminescent illumination with a camera system sensitive to the wavelength of emission of the bioluminescence system.

The invention further comprises a method of examining an animal, avian or human object, comprising the steps of providing a luciferase and a bioluminescence substrate, combining in the presence of any required activator the luciferase and the bioluminescence substrate to form a bioluminescent media, perfusing, administering, injecting, superfusing, infiltrating, or otherwise applying the mixture to a selected animal, avian or human object to be examined to provide bioluminescent illumination to the object and obtaining one or images of the object with bioluminescent illumination with a camera system sensitive to the wavelength of emission of the bioluminescent generating system.

In certain of the examination methods, the one or more images are obtained in dim visible light conditions.

In other of the examination methods, the one or more images are color or monochrome images.

In yet other of the examination methods, the inventive method further comprises obtaining one or more images of said object with visible light illumination, the visible light comprising visible light in a wavelength range which does not include light in a wavelength range of light emitted by the bioluminescent generating system. In some of these embodiments, the images are obtained with a camera system sensitive to the wavelength of emission of the visible light illumination. In certain of these embodiments, the visible light illumination is a red light illumination. In certain embodiments, the inventive method further comprises comparing the image with bioluminescent illumination with the image with visible light illumination.

In some of these embodiments, the method further comprises a step of switching between the image with bioluminescent illumination and said image with visible light illumination.

In some of the examination methods, the images are obtained by an endoscopic examination of the object.

In certain of the examination methods, the object comprises a component of an animal, avian or human circulatory system, nervous system, body organ, or lymphatic system. In some of these embodiments, the object comprises gallbladder, ureter, lung, heart, or intestine.

Other objects of the invention and its particular features and advantages will become more apparent from consideration of the following drawings and accompanying detailed description.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic illustration of the process of bioluminescence by oxidation of coelenterazine.

FIG. 2 is an illustration of the chemical structure of coelenterazine.

FIG. 3 is an illustration of the changes in the chemical structure of coelenterazine during bioluminescence.

FIG. 4 is a photograph showing a duodenal loop of a rat which has been administered with bioluminescent compounds in accordance with the present invention.

FIG. 5 is a photograph showing a swine gallbladder as viewed in visible light.

FIG. 6 is a photograph showing a swine gallbladder which has been administered with bioluminescent compounds in accordance with the present invention.

FIG. 7 is a monochrome photograph showing a swine gallbladder which has been administered with bioluminescent compounds in accordance with the present invention.

FIG. 8 is an inverse image monochrome photograph showing a swine gallbladder which has been administered with bioluminescent compounds in accordance with the present invention.

FIG. 9 is a photograph showing a swine bowel anastomosis as viewed in visible light.

FIG. 10 is a photograph showing a swine bowel anastomosis which has been administered with bioluminescent compounds in accordance with the present invention.

FIG. 11 is a photograph showing a swine lung as viewed in visible light.

FIG. 12 is a photograph showing a swine lung which has been administered with bioluminescent compounds in accordance with the present invention.

FIG. 13 is a photograph showing a swine heart as viewed in visible light.

FIG. 14 is a photograph showing a swine heart which has been administered with bioluminescent compounds in accordance with the present invention.

FIG. 15 is a photograph showing a swine small intestine as viewed in visible light.

FIG. 16 is a photograph showing a swine small intestine which has been administered with bioluminescent compounds in accordance with the present invention.

### DETAILED DESCRIPTION OF THE INVENTION

### A. Definitions

Unless defined otherwise, all technical and scientific terms used herein have the same meaning as is commonly understood by one of skill in the art to which this invention belongs. All patents and publications of referred to herein are incorporated by reference in their entirety.

As used herein, “chemiluminescence” refers to a chemical reaction in which energy is specifically channeled to a molecule causing it to become electronically excited and subsequently to release a photon thereby emitting visible light. Temperature does not contribute to this channeled energy. Thus, chemiluminescence involves the direct conversion of chemical energy to light energy.

As used herein, “luminescence” refers to the detectable electromagnetic radiation, generally, UV, IR or visible light radiation that is produced when the excited product of an exergic chemical process reverts to its ground state with the emission of light. Chemiluminescence is luminescence that results from a chemical reaction. Bioluminescence is chemiluminescence that results from a chemical reaction using biological molecules or synthetic versions or analogs thereof as substrates and/or enzymes.

As used herein, “bioluminescence,” which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein (luciferase) that is an oxygenase that acts on a substrate luciferin (a bioluminescence substrate) in the presence of molecular oxygen and transforms the substrate to an excited state, which upon return to a lower energy level releases the energy in the form of detectable electromagnetic radiation.

As used herein, the substrates and enzymes for producing bioluminescence are generically referred to as “luciferin” and “luciferase”, respectively. Activators are typically O₂, Mg⁺⁺, or Ca⁺⁺

“Luciferase” refers to oxygenases that catalyze a light emitting reaction. For instance, bacterial luciferases catalyze the oxidation of flavin mononucleotide [FMNI] and aliphatic aldehydes, which reaction produces light. Another class of luciferases, found among marine arthropods, catalyzes the oxidation of Cypridina (also known as Vargula) luciferin, and another class of luciferases catalyzes the oxidation of Coleoptera luciferin. Thus, luciferase refers to an enzyme or photoprotein that catalyzes a bioluminescent reaction (a reaction that produces bioluminescence). Luciferase enzymes such as firefly and Renilla luciferases act catalytically and are unchanged during the bioluminescence generating reaction. Luciferase photoproteins, such as the aequorin photoprotein to which luciferin is non-covalently bound, are changed, such as by release of the luciferin, during bioluminescence generating reaction. Luciferases employed in the present invention are proteins that occur naturally in an organism, and also variants or mutants thereof, such as a variant produced by mutagenesis that has one or more properties, such as thermal stability, that differ from the naturally-occurring protein. Luciferases and modified mutant or variant forms thereof are well known. For purposes of this application, reference to luciferase refers to either or both luciferase enzymes and photoproteins and their mutant, variant, and synthetic forms. Thus, reference, for example, to “Ranilla luciferase” means an enzyme isolated from member of the genus Renilla or an equivalent molecule obtained from any other source, such as from another Anthozoa, or that has been prepared synthetically.

As used herein, “bioluminescence substrate” refers to the compound that is oxidized in the presence of a luciferase, and any necessary activators, and generates light. These substrates, also referred to as “luciferin” or “luciferin solution” herein, are substrates that undergo oxidation in a bioluminescence reaction. These bioluminescence substrates include any luciferin or analog thereof or any synthetic compound with which a luciferase interacts to generate light. Preferred substrates are those that are oxidized in the presence of a luciferase or protein in a light-generating reaction. Bioluminescence substrates, thus, will typically consist of aqueous solutions of those compounds that those of skill in the art recognize as luciferins. Luciferins, for example, include firefly luciferin, Cypridina (also known as Vargula) luciferin (coelenterazine), bacterial luciferin, as well as synthetic analogs of these substrates or other compounds that are oxidized in the presence of a luciferase in a reaction the produces bioluminescence.

As used herein, “bioluminescence system” or “bioluminescence generating system” refers to the set of reagents required to conduct a bioluminescent reaction. Thus, the specific luciferase, luciferin and other substrates, activator, solvents and other reagents that may be required to complete a bioluminescent reaction form a bioluminescence system. Thus a bioluminescence system refers to any set of reagents that, under appropriate reaction conditions, yield bioluminescence. In general, bioluminescence systems include a bioluminescence substrate, luciferin, a luciferase, which includes enzyme luciferases and photoproteins, and one or more activators. A specific bioluminescence system may be identified by reference to the specific organism from which the luciferase derives; for example, the Vargula [also called Cypriciirla] bioluminescence system (or Vargula system) includes a Vargula luciferase, such as a luciferase isolated from the ostracod, Varguia or produced using recombinant means or modifications of these luciferases. This system would also include the particular activators necessary to complete the bioluminescence reaction, such as oxygen and a substrate with which the luciferase reacts in the presence of the oxygen to produce light.

“Appropriate reaction conditions” refers to the conditions necessary for a bioluminescence reaction to occur, such as pH, salt concentrations and temperature.

As used herein, a “surgical viewing” refers to any procedure in which an opening is made in the body of a subject. Such procedures include traditional human and animal surgeries and diagnostic procedures, such as but not limited to laparoscopy, thoracoscopy and arthroscopy procedures. Surgical viewing also refers to any procedure in which a natural orifice is accessed or obturated with a rigid or flexible scope such as but not limited to esophago-gastro-duodenoscopy, colonoscopy or bronchoscopy. Surgical viewing also refers to any angiography, venography, lymphangiography where vessels or tissue beds are cannulated or injected, such as but not limited to diagnostic mapping or completion intraoperative angiography for testing and verification of patency, integrity (leak), or arteriovenous fistula status. Surgical viewing also refers to open operations that do not typically employ an endoscope, rather benefit from a camera and open lens not inserted into a surgical opening or natural orifice, but positioned within close focal length of the operative field for anatomic structure identification. Surgical viewing also refers to open or micro-access operations employing a surgical microscope, such as but not limited to nervous system tumor resections.

Surgical viewing may range from but is not limited to the surgeon's naked eye, to the various endoscopes, open lens cameras and microscopes, and may also include use of various filters such as but not limited to a blue-green wavelength filter for the endoscope visualizing of a bioluminescence enhanced image and a red filter for selective background illumination of the operative field. Similarly, this technology may include any post image capture processing algorithms used to enhance the analog or digital images obtained, such as but not limited to processing by digital imaging computer software programs. This technology may range from but is not limited to simple refinement of the image, to fusion of 2 or more images in a composite still or movie format.

### B. Bioluminescence

Luminescence is a phenomenon in which energy is specifically channeled to a molecule to produce an excited state. Return to a lower energy state is accompanied by release of a photon (hv). Luminescence includes fluorescence, phosphorescence, chemiluminescence and bioluminescence. Bioluminescence is the process by which living organisms emit light that is visible to other organisms. Luminescence may be represented as follows:

A+B→X*+Y

X*_{→}X+hν

Where X* is an electronically excited molecule and hν represents light emission upon return of X* to a lower energy state. Where the luminescence is bioluminescence, creation of the excited state derives from an enzyme catalyzed reaction. The color of the emitted light in a bioluminescent (or chemiluminescent or other luminescent) reaction is characteristic of the excited molecule, and is independent from its source of excitation and temperature.

Though rare overall, bioluminescence is more common in marine organisms than in terrestrial organisms. Bioluminescence has developed from as many as thirty evolutionarily distinct origins and, thus, is manifested in a variety of ways so that the biochemical and physiological mechanisms responsible for bioluminescence in different organisms are distinct. Bioluminescent species span many genera and include microscopic organisms, such as bacteria (primarily marine bacteria including Vibrio species), fungi, algae and dinoflagellates, to marine organisms, including arthropods, mollusks, echinoderms, and chordates, and terrestrial organism including annelid worms and insects.

### C. Bioluminescence Generating Systems

A bioluminescence generating system includes the components that are necessary and sufficient to generate bioluminescence. These include a luciferase, luciferin and any necessary co-factors or conditions. Virtually any bioluminescent system known to those of skill in the art will be amenable to use in the methods provided herein.

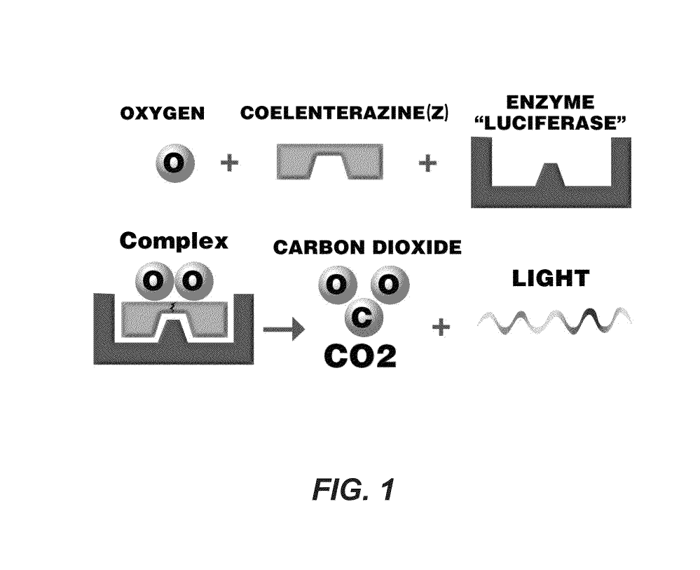

In general, bioluminescence refers to an energy-yielding chemical reaction in which a specific chemical substrate, a luciferin, undergoes oxidation, catalyzed by an enzyme, a luciferase. An essential condition for bioluminescence is the use of molecular oxygen, either bound or free in the presence of a luciferase. Luciferases, are oxygenases, which act on the substrate, luciferin, in the presence of molecular oxygen and transform the substrate to an excited state. Upon return to a lower energy level, energy is released in the form of light. This process is illustrated in FIG. 1: The oxidized reaction product is termed oxyluciferin, and certain luciferin precursors are termed etioluciferin. Thus, for purposes herein bioluminescence encompasses light produced by reactions that are catalyzed by (in the case of luciferases that act enzymatically) or initiated by (in the case of the photoproteins, such as aequorin, that are not regenerated in the reaction) a biological protein or analog, derivative or mutant thereof. Bioluminescent reactions are easily maintained, requiring only replenishment of exhausted luciferin or other substrate or cofactor or other protein, in order to continue or revive the reaction. Bioluminescence generating reactions are well-known to those of skill in this art and any such reaction may be adapted for use in combination with articles of manufacture as described herein.

Luciferases include enzymes such as the luciferases that catalyze the oxidation of luciferin, emitting light and releasing oxyluciferin. Also included among luciferases are photoproteins, which catalyze the oxidation of luciferin to emit light but are changed in the reaction and must be reconstituted to be used again. The luciferases may be naturally occurring or may be modified, such as by genetic engineering to improve or alter certain properties. As long as the resulting molecule retains the ability to catalyze the bioluminescent reaction, it is encompassed herein.

Any protein that has luciferase activity (catalysis of oxidation of a substrate in the presence of molecular oxygen to produce light as defined herein) may be used herein. The preferred luciferases are those that are described herein or that have minor sequence variations. Such minor sequence variations include, but are not limited to, minor allelic or species variations and insertions or deletions of residues, particularly cysteine residues. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially after biological activity. Other substitutions are also permissible and may be determined empirically or in accord with known conservative substitutions. Any such modification of the polypeptide may be effected by any means known to those of skill in this art.

It is understood that a bioluminescence generating system may be isolated from natural sources, or may be produced synthetically. In addition, for uses herein, the components need only be sufficiently pure so that mixture thereof, under appropriate reaction conditions, produces a glow so that cells and tissues can be visualized during a surgical procedure. Thus, in some embodiments, a crude extract or merely grinding up the organism may be adequate. Generally, however, substantially pure components are used. Also, components may be synthetic components that are not isolated from natural sources. DNA encoding luciferases is and synthetic and alternative substrates have been devised. Any bioluminescence generating system, whether synthetic or isolated from natural sources, is intended for use in the methods provided herein. The luciferases may be obtained commercially, isolated from natural sources, expressed in host cells using DNA encoding the luciferase, or obtained in any manner known to those of skill in the art. The luciferin substrates for the reaction or for inclusion in the conjugates include any molecule(s) with which the luciferase reacts to produce light. Such molecules include the naturally-occurring substrates, modified forms thereof, and synthetic analogues.

There are numerous organisms and sources of bioluminescence generating systems, and some representative genera and species that exhibit bioluminescence are set forth in Hastings, in (1995) Cell Physiology: Source Book, N. Sperelakis (ed.), Academic Press, pp 665-681]. Other bioluminescent organisms contemplated for use herein are Gonadostornias, Gaussia, Watensia, Halisturia, Vampire squid, Glyphus, Mycotophids (a fish), Vinciguerria, Howelia, Florenciella, Chaudiodus, Melanocostus and Sea Pens.

Examples of luciferases include, but are not limited to, those isolated from the ctenophores Mnemiopsis (mnemiopsin) and Beroe ovate (berovin), those isolated from the coelenterates Aequorea (aequorin), Obelia (obelin), Pelagia, the Renilla luciferase, the luciferases isolated from the mollusca Pholas (pholasin), the luciferases isolated from fish, such as Aristostornias, Pachystornias and Poriethys and from the ostracods, such as Cypridina (also referred to as Vargula).

The majority of commercial bioluminescence applications are based on firefly luciferase [Photinus pyralis]. One of the first and still widely used assays involves the use of firefly luciferase to detect the presence of ATP. It is also used to detect and quantify other substrates or co-factors in the reaction. Any reaction that produces or utilizes NAD(H). NADP(H) or long chain aldehyde, either directly or indirectly, can be coupled to the light-emitting reaction of bacterial luciferase.

Another luciferase system that has been used commercially for analytical purposes is the Aequorin photoprotein system. The purified jellyfish photoprotein, aequorin, is used to detect and quantify intracellular Ca and its changes under various experimental conditions. The Aequorin is relatively small [about 20 kDa], nontoxic, and can be injected into cells in quantities adequate to detect calcium over a large concentration range [3×10⁻⁷ to 10⁻⁴ M].

Because of theft analytical utility, many luciferases and substrates have been studied and well-characterized and are commercially available. Firefly luciferase is available from Sigma, St. Louis, Mo., and Boehringer Mannheim Biochemicals, Indianapolis, Ind.; recombinantly produced firefly luciferase and other reagents based on this gene or for use with this protein are available from Promega Corporation, Madison, Wis.; the aequorin photoprotein luciferase from jellyfish and luciferase from Renilla are commercially available from Prolume, Inc. (Pinetop, Ariz.); coelenterazine, the naturally-occurring substrate for these luciferases, is available from Invitrogen Molecular Probes (Carlsbad, Calif.) and Prolume, Inc. (Pinetop, Ariz.). These luciferases and related reagents are used as reagents for diagnostics, quality control, environmental testing and other such analyses.

Preferred luciferases for use herein are the Aequorin protein, Renilla luciferase and Cypridina (also called Vargula) luciferase. Also, preferred are luciferases which react to produce red and/or near infrared light. These include luciferases found in species of Aristostomias, such as A. scintillans, Pachysiornias, Malacosteus, such as M. niger.

The bioluminescent generating systems may also require additional components known to those of skill in the art. All bioluminescent reactions require molecular oxygen in the form of dissolved or bound oxygen. Thus, molecular oxygen, dissolved in water or in air or bound to a photoprotein, is the activator for bioluminescence reactions. Depending upon the form of the components, other activators include, but are not limited to, ATP (for firefly luciferase), flavin reductase for regenerating FMNH₂ from FMN (for bacterial systems), and Ca⁺⁺ or other suitable metal ions. While most of the systems provided herein will generate light when the luciferase and luciferin are mixed and exposed to air or water, the systems that use photoproteins that have bound oxygen, such as aequorin, will require exposure to Ca⁺⁺ or other suitable metal ion, which can be provided in the form of an aqueous composition of a calcium salt. In these instances, addition of a Ca⁺⁺ or other suitable metal on to a mixture of aequorin luciferase and coelenterazine luciferin will result in generation of light. The Renilla system and other Anthozoa systems also require Ca⁺⁺ other suitable metal ion.

Ctenophores, such as Mnemiopsis (mnemiopsin) and Beroe ovate (berovin), and coelenterates, such as Aequorea (aequorin), Obelia (obelin) and Pelagia, produce bioluminescent light using similar chemistry. The Aequorin and Renilla systems are representative and are described in detail herein as exemplary and as among the presently preferred systems. The Aequorin and Renilia systems can use the same luciferin and produce light using the same chemistry, but each luciferase is different.

The Aequorin luciferase aequorin, as well as, for example, the luciferases mnemiopsin and berovin, is a photoprotein that includes bound oxygen and bound luciferin, requires Ca⁺⁺ (or other suitable metal ion) to trigger the reaction, and must be regenerated for repeated use. The Renilla luciferase also benefits from Ca⁺⁺ or other suitable metal ion but acts as a true enzyme because it is unchanged during the reaction and it requires dissolved molecular oxygen. See, e.g., Allen, D. G., J. R. Blinks, et al. (1977) “Aequorin luminescence: relation of light emission to calcium concentration—a calcium-independent component.” Science 195(4282): 996-8; Blinks, j. R., F. G. Prendergast, et al. (1976) “Photoproteins as biological calcium indicators.” Pharmacological Reviews 28(1): 1-93.; Charbonneau, H., K. A. Walsh, et al. (1985). “Amino acid sequence of the calcium-dependent photoprotein aequorin.” Biochemistry 24(24): 6762-71; Cormier, M. J., D. C. Prasher, et al. (1989). “The enzymology and molecular biology of the Ca²⁺ activated photoprotein, aequorin.” Photochemistry & Photobiology 49(4): 509-12.; Hannick, L. I., D. C. Prasher, et al. (1993). “Preparation and initial characterization of crystals Inouye, S. (2007). “Expression, purification and characterization of calcium-triggered luciferin-binding protein of Renilla reniformis.” Protein Ex ession & Purification 52(1): 66-73.; Inouye, S, and Y. Sahara (2008). “Identification of two catalytic domains in a luciferase secreted by the copepod Gaussia princeps.” Biochemical & Biophysical Research Communications 365(1): 96-101; Inouye, S, and S. Sasaki (2006). “Blue fluorescent protein from the calcium-sensitive photoprotein aequorin: catalytic properties for the oxidation of coelenterazine as an oxygenase.” FEBS Letters 580(8): 1977-82.; Inouye, S, and S. Sasaki (2007). “Imidazole-assisted catalysis of luminescence reaction in blue fluorescent protein from the photoprotein aequorin.” Biochemical & Biophysical Research Communications 354(3): 650-5.; Inouye, S, and O, Shimomura (1997). “The use of Renilla luciferase, Oplophorus luciferase, and apoaequorin as bioluminescent reporter protein in the presence of coelenterazine analogues as substrate.” Biochemical & Biophysical Research Communications 233(2): 349-53.; Inouye, S., K. Watanabe, at al. (2000). “Secretional luciferase of the luminous shrimp Oplophorus gracilirostris: cDNA cloning of a novel imidazopyrazinone luciferase(1).” FEBS Letters 481(1): 19-25.; Liu, Z. J., G. A. Stepanyuk, et al. (2006). “Crystal structure of obelin after Ca2+-triggered bioluminescence suggests neutral coelenteramide as the primary excited state.” Proceedings of the National Academy of Sciences of the United States of America 103(8): 2570-5.; Loaning, A. M., T. D. Fenn, et al. (2007). “Crystal structures of the luciferase and green fluorescent protein from Renilia reniformis.” Journal of Molecular Biology 374(4): 1017-28.; Loening, A. M., T. D. Fenn, et al. (2006). “Consensus guided mutagenesis of Renilla luciferase yields enhanced stability and light output.” Protein Engineering, Design & Selection 19(9): 391-400.; Loening, A. M., A. M. Wu, et al. (2007). “Red-shifted Renilia reniformis luciferase variants for imaging in living subjects. [see comment].” Nature Methods 4(8): 641-3.; Otto-Duessel, M., V. Khankaldyyan, et al. (2006). “In vivo testing of Renilla luciferase substrate analogs in an orthotopic murine model of human glioblastoma.” Molecular Imaging: Official Journal of the Society for Molecular Imaging 5(2); 57-64.; Prasher, D. C., R. O. McCann, et al. (1987). “Sequence comparisons of complementary DNAs encoding aequorin isotypes.” Biochemistry 26(5): 1326-32.18.; Ray, B. D., S. Ho, et al. (1985). “Proton NMR of aequorin. Structural changes concomitant with calcium-independent light emission.” Biochemistry 24(16): 4280-7.; Shimomura, O. (1986). “Isolation and properties of various molecular forms of aequorin.” Biochemical Journal 234(2): 271-7.; Shimomura, O. (1995). “Cause of spectral variation in the luminescence of semisynthetic aequorins.” Biochemical Journal 306(Pt 2): 537-43.; Shimomura, O. (1995). “Luminescence of aequorin is triggered by the binding of two calcium ions.” Biochemical & Biophysical Research Communications 211(2): 359-63.; Shimomura, O. (1995). “A short story of aequorin.” Biological Bulletin 189(1): 1-5.; Shimomura, O. (1997). “Membrane permeability of coelenterazine analogues measured with fish eggs.” Biochemical Journal 326(Pt 2): 297-8.; Shimomura, O. (2005). “The discovery of aequorin and green fluorescent protein.” Journal of Microscopy 217(Pt 1): 1-15.; Shimomura, O., P. R. Flood, et al. (2001). “Isolation and properties of the luciferase stored in the ovary of the scyphozoan medusa Periphylla periphylia.” Biological Bulletin 201(3): 339-47.; Shimomura, O. and S. Inouye (1999). “The in situ regeneration and extraction of recombinant aequorin from Escherichia coli cells and the purification of extracted aequorin.” Protein Expression & Purification 16(1): 91-5.; Shimomura, O. and F. H. Johnson (1969). “Properties of the bioluminescent protein aequorin.” Biochemistry 8(10): 3991-7.; Shimomura, O. and F. H. Johnson (1970). “Mechanisms in the quantum yield of Cypridina bioluminescence.” Photochemistry & Photobiology 12(4): 291-5.; Shimomura, O. and F. H. Johnson (1971). “Mechanism of the luminescent oxidation of cypridina luciferin.” Biochemical & Biophysical Research Communications 44(2): 340-6.; Shimomura, O. and F. H. Johnson (1972). “Structure of the light-emitting moiety of aequorin.” Biochemistry 11(9): 1602-8.; Shimomura, O. and F. H. Johnson (1973). “Further data on the specificity of aequorin luminescence to calcium.” Biochemical & Biophysical Research Communications 53(2): 490-4.; Shimomura, O. and F. H. Johnson (1975). “Chemical nature of bioluminescence systems in coelenterates.” Proceedings of the National Academy of Sciences of the United States of America 72(4): 1546-9.; Shirnomura, O. and F. H. Johnson (1975). “Regeneration of the photoprotein aequorin.” Nature 256(5514): 236-8; Shimomura, O. and F. H. Johnson (1978). “Peroxidized coelenterazine, the active group in the photoprotein aequorin.” Proceeding of the National Academy of Sciences of the United States of America 75(6): 2611-5.; Shirnomura, O., F. H. Johnson, et al. (1974). “Mechanism of the luminescent intramolecular reaction of aequorin.” Biochemistry 13(16): 3278-86.; Shimomura, O., F. H. Johnson, at al. (1961). “Purification and properties of Cypridina luciferase.” Journal of Cellular & Comparative Physiology 58: 113-23.; Shirnomura, O., Y. Kishi, at al. (1993). “The relative rate of aequorin regeneration from apoaequorin and coelenterazine analogues,” Biochemical Journal 296(Pt 3): 549-51.; Shimomura, O., T. Masugi, et al. (1978). “Properties and reaction mechanism of the bioluminescence system of the deep-sea shrimp Oplophorus grachlorostris.” Biochemistry 17(6): 994-8.; Shimomura, O., B. Musicki, et al. (1989). “Semi-synthetic aequorins with improved sensitivity to Ca2+ ions.” Biochemical Journal 261(3): 913-20.; Shimomura, O., B. Musicki, et al. (1993). “Light-emitting properties of recombinant semi-synthetic aequorins and recombinant fluorescein-conjugated aequorin for measuring cellular calcium.” Cell Calcium 14(5): 373-8.; Shimomura, O. and A. Shimomura (1981). “Resistivity to denaturation of the apoprotein of aequorin and reconstitution of the luminescent photoprotein from the partially denatured apoprotein.” Biochemical Journal 199(3): 825-8.; Shimomura, O. and K. Teranishi (2000). “Light-emitters involved in the luminescence of coelenterazine.” Luminescence 15(1): 51-8.; Shimomura, O., C. Wu, et al. (1998), “Evaluation of five imidazopyrazinone-type chemiluminescent superoxide probes and their application to the measurement of superoxide anion generated by Listeria monocytogenes.” Analytical Biochemistry 258(2): 230-5.

This system is among the preferred systems for use herein. As will be evident, since the aequorin photoprotein includes noncovalently bound luciferin and molecular oxygen, it is suitable for storage in this form as a lyophilized powder or encapsulated into a selected delivery vehicle. The system can be encapsulated into pellets, such as liposomes or other delivery vehicles. When used, the vehicles are contacted with a composition, even tap water that contains Ca⁺⁺ or other suitable metal ion, to produce a mixture that glows.

It is also understood that these mixtures will also contain any additional salts or buffers or ions that are necessary for each reaction to proceed. Since these reactions are well-characterized, those of skill in the art will be able to determine precise proportions and requisite components. Selection of components will depend upon the apparatus, article of manufacture and luciferase. Factors for consideration in selecting a bioluminescent-generating system, include, but are not limited to: the targeting agent used in combination with the bioluminescence; the medium in which the reaction is run; stability of the components, such as temperature or pH sensitivity; shelf life of the components; sustainability of the light emission, whether constant or intermittent; availability of components; desired light intensity; color of the light; and other such factors.

### D. Methods of Use

In most embodiments, an aqueous luciferin solution (bioluminescence substrate) will be formulated with any other desired components (including an activator), and kept separate from its related aqueous luciferase solution. When ready for use, the luciferin solution and the luciferase solution are combined ex vivo to initiate bioluminescence and injected into or applied to the organ or tissue to be illuminated.

The bioluminescent generating system components are combined ex vivo immediately prior to injection or infusion into a tissue area of interest to enable bioluminescent viewing of the tissue. The bioluminescent generating system components are combined by simultaneous injection into a Y tube or other in-line mixing chamber receiving fluid inputs from two or more fluid conduits and providing a fluid output to at least one fluid conduit. The mixed components which are the output of such multi lumen mixing system is directed to or injected into a hollow viscus organ or otherwise applied to organs and/or tissues of the body

The glow should be sufficient to see under dim visible light or, if necessary, in the dark. This is typical but not limited to the bioluminescent cholangiography and bowel anastomotic patency testing methods described herein, whereby the duct or hollow viscus is accessed with an angio-catheter or similar catheter or cannula which is connected to a short length of a Y-tubing which is used to infuse the bioluminescence generating system (or luciferin and luciferase). Surgical viewing may be by naked eye or endoscopic methods as described. For navigational or diagnostic mapping applications intra-operative administration of the bioluminescent agent is via any suitable route, whereby the agent fills and illuminates the tubes, ducts, lumens, chambers, vessels or hollow organs.

In general, since the result to be achieved is the production of light visible to the naked eye for qualitative, not quantitative, diagnostic purposes, the precise proportions and amounts of components of the bioluminescence reaction need not be stringently determined or met. They must be sufficient to produce light. Generally, an amount of luciferin and luciferase sufficient to generate a visible glow is used; this amount can be readily determined empirically and is dependent upon the selected system and selected application. Where quantitative measurements are required, more precision may be required.

Higher concentrations may be used if the glow is not sufficiently bright. Alternatively, a microcarrier coupled to more than one luciferase molecule linked to a targeting agent may be utilized to increase signal output. Also because the conditions in which the reactions are used are not laboratory conditions, and the components are subject to storage, higher concentration may be used to overcome any loss of activity.

### E. Reaction Mixture Formulations

Luciferase for use in accordance with the invention will be provided in an aqueous solution at a concentration of between about 0.01 mg and 100 mg per liter of reaction mixture (the total of all components of the bioluminescence mixture). In embodiments in which the luciferase acts catalytically and does not need to be regenerated, lower amounts of luciferase can be used. In those in which it is changed during the reaction, it also can be replenished; typically higher concentrations will be selected. In most typical applications, the luciferase will be provided at a concentration of 0.1 to 20 mg, preferably 0.1 to 10 mg, more preferably between about 1 and 10 mg per liter of reaction mixture. Concentrations of at least 1 mg or more are preferred for a brighter result.

Luciferin will be provided in an aqueous solution at a concentration of between about 0.01 mg and 100 mg per liter of reaction mixture, and in most typical applications, the luciferin will be provided at a concentration of 0.1 to 20 mg, preferably 0.1 to 10 mg, more preferably between about 1 and 10 mg per liter of reaction mixture. Concentrations of at least 1 mg or more are preferred. Additional luciferin can be added to many of the reactions to continue the reaction. When preparing coated substrates, coating compositions containing higher concentrations of the luciferase or luciferin may be used.

The reaction mixture will contain additional ingredients as needed to enhance viscosity, adhesion, or to activate the bioluminescent reaction may be included in amounts from about 0.01 mg/l, to about 10 mg/l or more of the reaction mixture. This can include but is not limited to polyethylene glycols of molecular weights from 400 to 20,000; water-soluble cellulose esters such as methylcellulose, ethylcellulose, carboxyethyl cellulose, carboxymethyl celluose; bacterially-derived carbohydrates such as dextran and beta-cyclodextrin, as well as chemically-modified cyclodextrins well known to those skilled in the art; chemically-modified starches; albumin proteins including ovalbumin, human serum albumin, bovine serum albumin, canine serum albumin, and feline serum albumin; simple alcohols such as glycerol, 1,3-propanediol, and ethanol; carbohydrate alcohols such as sorbitol, mannitol, pentaerythreitol, and water soluble esters thereof; non-reducing sugars, biologically-compatible fluorinated compounds which increase oxygen transport, including but not limited to perfluorotributylamine, perfluorodecalin, and perfluoroalcohols and their esters; solubilizers and nonionic and zwiterionic detergents including but not limited to the Tween, Pluronic, Triton series and cholic acid, sodium desoxycholate, CHAPS (Cholamidopropanesulfonate), biologically compatible organic buffers typified by but not limited to TRIS, BIS-TRIS, HEPES, MES, and inorganic ions typified by but not limited to Calcium ion (Ca⁺⁺).

Thus, for example, 5 mg of luciferin, such as coelenterazine, in one liter of water will glow brightly for at least about 10 to 20 minutes, depending on the temperature of the water, when about 10 mgs of luciferase, such as aequorin photoprotein luciferase or luciferase from Renilla, is added thereto in presence of Ca⁺⁺. Increasing the concentration of luciferase, for example, to 100 mg/L, provides a particularly brilliant light display.

It is understood, that concentrations and amounts to be used depend upon the selected bioluminescence generating system but these may be readily determined empirically. Proportions, particularly those used when commencing an empirical determination, are generally those used for analytical purposes, and amounts or concentrations are at least those used for analytical purposes, but the amounts can be increased, particularly if a sustained and brighter glow is desired.

### F. Aequorin Systems

The bioluminescence photoprotein aequorin is isolated from a number of species of the jellyfish Aequorea. It is a 22 kilodalton [kDa] molecular weight peptide complex. The native protein contains oxygen and a heterocyclic compound coelenierazine, a luciferin, noncovalently bound thereto. The protein contains three calcium binding sites. Upon addition of trace amounts Ca⁺⁺ or other suitable metal ion, such as strontium to the photoprotein, it undergoes a conformational change that catalyzes the oxidation of the bound coelenterazine using the protein-bound oxygen. Luminescence is triggered by calcium, which releases oxygen and the luciferin substrate producing apoaequorin. Energy from this oxidation is released as a flash of blue light, centered at 469 nm. Concentrations of calcium ions as low as 10⁻⁶ M are sufficient to trigger the oxidation reaction. Aequorin does not require dissolved oxygen.

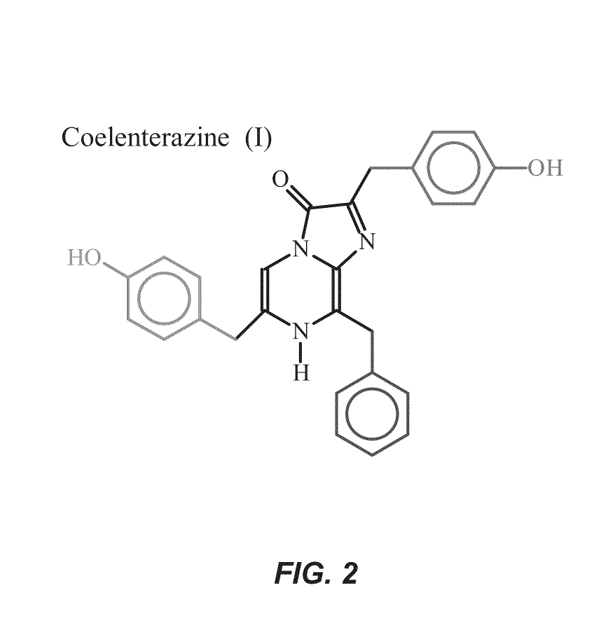

The aequorin luciferin coelenterazine is a molecule having Structure I shown in FIG. 2, and analogs and sulfated derivatives thereof.

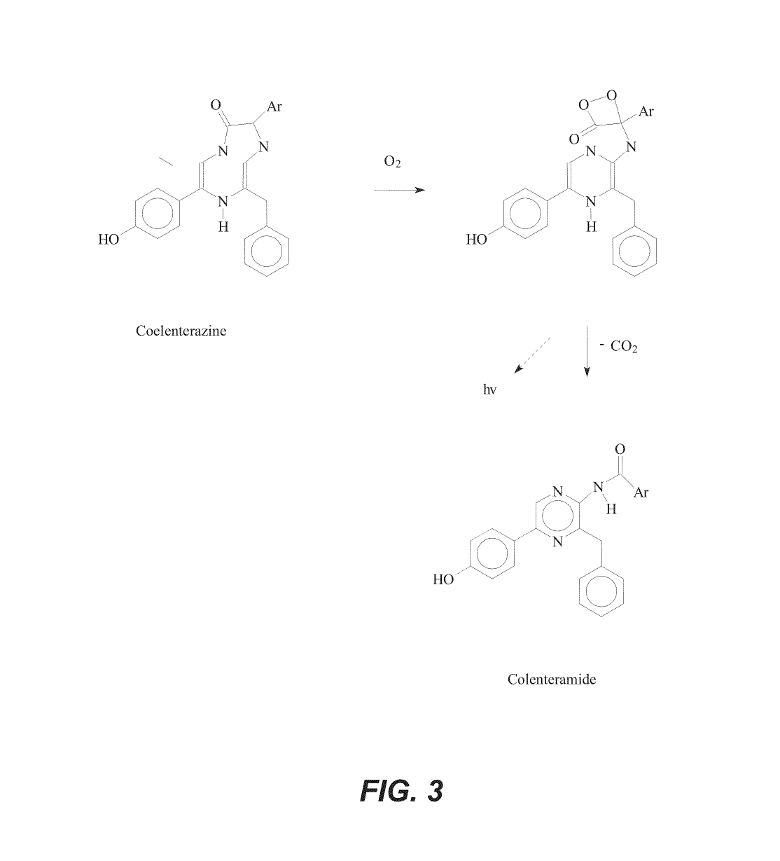

The reaction of coelenterazine when bound to the aequorin photoprotein with bound oxygen and in the presence of Ca⁺⁺ is shown in FIG. 3.

Naturally-occurring apoaequorin is not a single compound but rather is a mixture of microheterogeneous molecular species. Aequoria jellyfish extracts contain as many as twelve distinct variants of the protein. DNA encoding numerous forms has been isolated.

Numerous isoforms of the aequorin apoprotein been isolated. DNA encoding these proteins has been cloned, and the proteins and modified forms thereof have been produced using suitable host cells. DNA encoding apoaeguorin or variants thereof is useful for recombinant production of high quantities of the apoprotein. The preferred aequorin is produced using DNA, and known to those of skill in the art or modified forms thereof. The DNA encoding aequorin is expressed in a host cell, such as E. coli, isolated and reconstituted to produce the photoprotein. Of interest herein are forms of the apoprotein that have been modified so that the bioluminescent activity is greater than unmodified apoaequorin.

The photoprotein can be reconstituted by combining the apoprotein, such as a protein recombinantly produced in E. coli, with the luciferin, coelenterazine (preferably a sulfated derivative thereof, or an analog thereof), such as a synthetic coelenterazine, in the presence of molecular oxygen and a reducing agent such as 2-mercaptoethanol, and also EDTA or EGTA to tie up any Ca⁺⁺ to prevent triggering the oxidation reaction until desired. (DNA encoding a modified form of the apoprotein that does not require 2-mercaptoethanol for reconstitution is also possible). The constituents of the bioluminescence generating reaction can be mixed under appropriate conditions to regenerate the photoprotein and concomitantly have the photoprotein produce light.

For use in certain embodiments herein, the apoprotein and other components of the aequorin bioluminescence generating system are packaged or provided as a mixture, which, when desired, is subjected to conditions under which the photoprotein reconstitutes from the apoprotein, luciferin and oxygen. Particularly preferred are forms of the apoprotein that do not require a reducing agent, such as 2-mercaptoethanol, for reconstitution, The photoproteins and luciferases from related species, such as (Melia are also contemplated for use herein. DNA encoding the Calcium-activated photoprotein obelin from the hydroid polyp Obelia longissima is known and available [see, e.g., Deng, L., S. V. Markova, et al, (2004). “Preparation and X-ray crystallographic analysis of the Ca2+-discharged photoprotein obelin.” Acta Crystallographica Section D-Biological Crystallography 60(Pt 3): 512-41. In general for use herein, the components of the bioluminescence are packaged or provided so that there is insufficient metal ions to trigger the reaction. When used, the trace amounts of triggering metal ion, particularly Ca⁺⁺ is contacted with the other components. For a more sustained glow, aequorin can be continuously reconstituted or can be added or can be provided in high excess.

The light reaction is triggered by adding Ca⁺⁺ at a concentration sufficient to overcome the effects of the chelator and achieve the 10⁻⁶ M concentration. Because such low concentrations of Ca⁺⁺ can trigger the reaction, for use in the methods herein, higher concentrations of chelator may be included in the compositions of photoprotein. Accordingly, higher concentrations of added Ca⁺⁺ in the form of a calcium salt will be required. Precise amounts may be empirically determined. For use herein, it may be sufficient to merely add water to the photoprotein, which is provided in the form of a concentrated composition or in lyophilized or powdered form. Thus, for purposes herein, addition of small quantities of Ca⁺⁺, such as those present in phosphate buffered saline (PBS) or other suitable buffers or the moisture on the tissue to which the compositions are contacted, should trigger the bioluminescence reaction.

The photoprotein aequorin, which contains apoaequorin bound to a coelenterate luciferin molecule, and Renilla luciferase, can use the same coelenterate luciferin. The aequorin photoprotein catalyses the oxidation of coelenterate luciferin [coelenterazine] to oxyluciferin [coelenteramide] with the concomitant production of blue light [lambda max=469 nm]. (See FIG. 3).

The sulfate derivative of the coelenterate luciferin [lauryl-luciferin] is particularly stable in water, and thus may be used in a coelenterate-like bioluminescent system. In this system, adenosine diphosphate (ADP) and a sulphakinase are used to convert the coelenterazine to the sulphated form. Sulfatase is then used to reconvert the lauryl-luciferin to the native coelenterazine. Thus, the more stable lauryl-luciferin is used in the item to be illuminated and the luciferase combined with the sulfatase are added to the luciferin mixture when illumination is desired.

The bioluminescent system of Aequorea is particularly suitable for use in the methods herein. The particular amounts and the manner in which the components are provided depend upon the type of neoplasia or specialty tissue to be visualized. This system can be provided in lyophilized form, which will glow upon addition of Ca++. It can be encapsulated, linked to microcarriers, such as microbeads, or in as a compositions, such as a solution or suspension, preferably in the presence of sufficient chelating agent to prevent triggering the reaction. The concentration of the aequorin photoprotein will vary and can be determined empirically. Typically concentrations of at least 0.1 mg/l, more preferably at least 1 Mail and higher, will be selected. In certain embodiments. 1-10 mg luciferin/100 mg of luciferase will be used.

### G. Renilla Systems

The Renilla system is representative of coelenterate bioluminesence systems. Renilla, also known as sea pansies, are members of the class of coelenterates Anthozoa, which includes other bioluminescent genera, such as Cavarnularia, Ptilosarcus, Stylatula, Acanthoptilum, and Parazoanthus. Bioluminescent members of the Anthozoa genera contain luciferases and luciferins that are similar in structure [see, e.g., Cormier et al. (1973) J. Cell. Physiol. 81:291-298; see, also Ward et al. (1975) Proc. Natl. Acad. Sci. U.S.A. 72:2530-2534]. The luciferases and luciferins from each of these anthozoans crossreact with one another and produce a characteristic blue luminescence.

Renilia luciferase and the other coelenterate and ctenophore luciferases, such as the aequorin photoprotein, use imidazopyrazine substrates, particularly the substrates generically called coelenterazine [see, Formula I (FIG. 2) above]. Other genera that have luciferases that use a coelenterazine include: squid, such as Chiroieuthis, Eucieoteuthis, Onychoteuthis, Watasenia, cuttlefish, Sepiolina; shrimp, such as Oplophorus, Acanthophyra, Sergestes, and Gnathophausia; deep-sea fish, such as Argyropelecus, Yarella, Diaphus, Gonadostomias and Neoscopelus. Renilla luciferase does not, however, have bound oxygen, and thus requires dissolved oxygen in order to produce light in the presence of a suitable luciferin substrate. Since Renilla luciferase acts as a true enzyme, i.e., it does not have to be reconstituted for further use, the resulting luminescence can be long-lasting in the presence of saturating levels of luciferin. Also, Renilla luciferase is relatively stable to heat. Renilia luciferase, DNA encoding Renilia luciferase, and use of the DNA to produce recombinant luciferase, as well as DNA encoding luciferase from other coelenterates, are well known, (See for example Bhaumik, S., X. Z. Lewis, et al. (2004). “Optical imaging of Renilla luciferase, synthetic Renilla luciferase, and firefly luciferase reporter gene expression in living mice.” Journal of Biomedical Optics 9(3): 578-86.; Inouye, S. and O. Shimomura (1997). “The use of Renilla luciferase, Opiophorus luciferase, and apoaequorin as bioluminescent reporter protein in the presence of coelenterazine analogues as substrate.” Biochemical & Biophysical Research Communications 233(2): 349-53.; Loening, A. M., T. D. Fenn, et al. (2007). “Crystal structures of the luciferase and green fluorescent protein from Renilla reniformis.” Journal of Molecular Biology 374(4): 1017-28. Loening, A. M., T. D. Fenn, et al. (2006). “Consensus guided mutagenesis of Renilla luciferase yields enhanced stability and light output” Protein Engineering, Design & Selection 19(9): 391-400.; Loaning, A. M., A. M. Wu, et al. (2007). “Red-shifted Renilla reniformis luciferase variants for imaging in living subjects.” Nature Methods 4(8): 641-3.)

The DNA encoding Renilla luciferase and host cells containing such DNA provide a convenient means for producing large quantities of the enzyme. When used herein, the Renilla luciferase can be packaged in lyophilized form, encapsulated in a vehicle, either by itself or in combination with the luciferin substrate. Prior to use the mixture is contacted with an aqueous composition, preferably a phosphate buffered saline pH 7-8; dissolved O₂ will activate the reaction. Final concentrations of luciferase in the glowing mixture will be on the order of 0.01 to 1 mg/l or more. Concentrations of luciferin will be at least about 10⁻⁸ M, but preferably are 1 to 100 or more orders of magnitude higher to produce a long lasting bioluminescence.

In certain embodiments herein, about 1 to 10 mg, or preferably 2-5 mg, more preferably about 3 mg of coelenterazine will be used with about 100 mg of Renilla luciferase. The precise amounts, of course can be determined empirically, and, also will depend to some extent on the ultimate concentration and application. In one example, the addition of about 0.25 ml of a crude extract from the bacteria that express Renilia to 100 ml of a suitable assay buffer and about 0.005 mg of coelenterazine was sufficient to produce a visible and lasting glow.

Lyophilized mixtures, and compositions containing the Renilla luciferase are also provided. The luciferase or mixtures of the luciferase and luciferin may also be encapsulated into a suitable delivery vehicle, such as a liposome, glass particle, capillary tube, drug delivery vehicle, gelatin, time release coating or other such vehicle. The luciferase may also be linked to a substrate, such as biocompatible materials.

### G. Crustacean, Particularly Cyrpidina Systems

The ostracods, such as Vargula serratta, V. hilgendorfii and V. noctiluca are small marine crustaceans, sometimes called sea fireflies. These sea fireflies are found in the waters off the coast of Japan and emit light by squirting luciferin and luciferase into the water, where the reaction, which produces a bright blue luminous cloud, occurs. The reaction involves only luciferin, luciferase and molecular oxygen, and, thus, is very suitable for application herein.

The systems, such as the Vargula bioluminescent systems, are particularly preferred herein because the components are stable at room temperature if dried and powdered and will continue to react even if contaminated. Further, the bioluminescent reaction requires only the luciferin/luciferase components in concentrations as low as 1:40 parts per billion to 1:100 parts per billion, water and molecular oxygen to proceed. Importantly an exhausted system can renew by addition of luciferin.

Vargula luciferase is water soluble and is among those preferred for use in the methods herein. Varguia luciferase is a 555-amino acid polypeptide that has been produced by isolation from Vargula and also using recombinant technology by expressing the DNA in suitable bacterial and mammalian hosts.

Methods for purification of Vargula [also known as Cypridina] luciferase are well known. For example, crude extracts containing the active can be readily prepared by homogenizing or crushing the Vargula shrimp. In other embodiments, a preparation of Cypridina hilgendorfii luciferase CaO be prepared by immersing stored frozen C. hilgendorfii in distilled water containing, 0.5-5.0 M salt, preferably 0.5-2.0 M sodium or potassium chloride, ammonium sulfate, at 0-30° C. preferably 0-10° C., for 1-48 hr, preferably 10-24 hr, for extraction followed by hydrophobic chromatography and then ion exchange or affinity chromatography.

The luciferin can be isolated from ground freeze-dried Vargula by heating the extract, which destroys the luciferase but leaves the iuciferin intact

Varguia [also known as Cypridina] luciferase is preferably produced by expression of cloned DNA encoding the luciferase. DNA encoding the luciferase or variants thereof is introduced into E. coli using appropriate vectors and isolated using standard methods.

Natural Vargula [also known as Cypridina] luciferase has a substituted imidazopyrazine nucleus. Analogs thereof well known in the prior art and other compounds that react with the luciferin in a light producing reaction also may be used. Other bioluminescent organisms that have luciferases that can react with the Vargula luciferin include, the genera Apogon, Parapriacanthus and Porichthys.

The luciferin upon reaction with oxygen forms a dioxetanone intermediate [which includes a cyclic peroxide similar to the firefly cyclic peroxide molecule intermediate]. In the final step of the bioluminescent reaction, the peroxide breaks down to form CO₂ and a molecule with the C═O bond in an electronically excited state. The excited state molecule then returns to the ground state and in this process emits a blue to blue-green light.

The optimum pH for the reaction is about 7. For purposes herein, any pH at which the reaction occurs may be used. The concentrations of reagents are those normally used for analytical reactions or higher [see, e.g., Thompson et al. (1990) Gene 96:257-262]. Typically concentrations of the luciferase between 0.1 and 10 mg/l, preferably 0.5 to 2.5 mg/l will be used, Similar concentrations or higher concentrations of the luciferin may be used.

### H. Other Fluorescent Protein Systems

Blue light is produced using the Renilia luciferase or the Aequorea photoprotein in the presence of Ca⁺⁺ and the coelenterazine luciferin or analog thereof. By means of Dexter-Forster energy transfer, this light can be converted into a light of a different and longer wavelength if a green fluorescent protein (GFP) is added to the reaction. Green fluorescent proteins, which have been purified and also are used by cnidarians as energy-transfer acceptors. GFPs fluoresce in vivo upon receiving energy from a luciferase-oxyluciferein excited-state complex or a Ca⁺⁺-activated photoprotein. This process is known as Bioluminescent Resonant Energy Transfer (BRET) and has been utilized extensively for a wide variety of biological assay systems. In GFP, the chromophore is series of adjacent modified amino acid residues within the polypeptide. The best characterized GFPs are those of Aequorea and Renilia. For example, a green fluorescent protein from Aequorea Victoria contains 238 amino acids, absorbs blue light and emits green light. Thus, inclusion of this protein in a composition containing the aequorin photoprotein charged with coelenterazine and oxygen, can, in the presence of calcium, result in the production of green light. Thus, it is contemplated that GFPs may be included in the bioluminescence generating reactions that employ the aequorin or Renilia luciferases or other suitable luciferase in order to enhance or alter color of the resulting bioluminescence. Many genetically-altered GFPs are well known in the prior art, and these can produce colors from the blue to the red.

GFPs are activated by blue light to emit green light and thus may be used in the absence of luciferase and in conjunction with an external light source to illuminate neoplasia and specialty tissues, as described herein.

Similarly, blue fluorescent proteins (BFPs), such as from Vibrio fischeri, Vibrio harveyi or Photobacterium phosphoreum, may be used in conjunction with an external light source of appropriate wavelength to generate blue light. In particular, GFPs, and/or BFPs or other such fluorescent proteins may be used in the methods described herein using a targeting agent conjugate by illuminating the conjugate with light of an appropriate wavelength to cause the fluorescent proteins to fluoresce.

Such systems are particularly of interest because no luciferase is needed to activate the photoprotein. These fluorescent proteins may also be used in addition to bioluminescence generating systems to enhance or create an array of different colors.

### I. Phycobiliprotein Systems

Phycobiliproteins are water soluble fluorescent proteins derived from cyanobacteria. These proteins have been used as fluorescent labels in immunoassays; the proteins have been isolated and DNA encoding them is also available; the proteins are commercially available from, for example, ProZyme, Inc., San Leandro, Calif.

In these organisms, the Phycobiliproteins are arranged in subceilular structures termed phycobilisomes, and function as accessory pigments that participate in photosynthetic reactions by absorbing visible light and transferring the derived energy to chlorophyll via a direct fluorescence energy transfer mechanism.

Two classes of phycobiliproteins are known based on their color: phycoerythrins (red) and phycocyanins (blue), which have reported absorption maximal between 490 and 570 nm and between 610 and 665 nm, respectively. Phycoerythrins and phycocyanins are heterogeneous complexes composed of different ratios of alpha and beta monomers to which one or more class of linear tetrapyrrole chromophores are covalently bound. Particular phycobiliproteins may also contain a third subunit which often associated with aggregate proteins.

All phycobiliproteins contain either phycothrombilin or phycoerythobilin chromophores, and may also contain other bilins phycourobilin, crypioviolin or the 697 nm bilin. The subunit is covalenily bound with phycourobilin which results in the 495-500 nm absorption peak of B- and R-phycoerythrins. Thus, the spectral characteristics of phycobiliproteins may be influenced by the combination of the different chromophores, the subunit composition of the apophycobiliproteins and/or the local environment effecting the tertiary and quaternary structure of the phycobiliproteins.

As described above for GFPs and BFPs, phycobiliproteins are also activated by visible light of the appropriate wavelength and, thus, may be used in the absence of luciferase and in conjunction with an external light source to illuminate neoplasia and specialty tissues, as described herein. These proteins may be used in combination with other fluorescent proteins and/or bioluminescence generating systems to produce an array of colors or to provide different colors over time. Attachment of phycobiliproteins to solid support matrices is known. Therefore, phycobiliproteins may be coupled to microcarriers coupled to one or more components of the bioluminescent reaction, preferably a luciferase, to convert the wavelength of the light generated from the bioluminescent reaction. Microcarriers coupled to one or more phycobiliproteins may be used in any of the methods provided herein.

The conversion of blue or green light to light of a longer wavelength, i.e., red or near infra-red, is particularly preferred for the visualization of deep neoplasias or specialty tissues using a laparoscope or computer tomogram imaging system. Thus, when a change in the frequency of emitted light is desired, the phycobiliprotein, or other spectral shifter, such as synthetic fluorochrome, green fluorescent proteins, red fluorescent proteins, and substrates altered chemically or enzymatically to cause shifts in frequency of emission can be included with the bioluminescent generating components.

### J. Use of Bioluminescence Generating Systems on Test Subjects

Instillation of a bioluminescent solution into the bile duct, intestinal anastomosis, or ureter during surgery allows excellent instantaneous visualization to the surgeon, potentially preventing damage to these structures. These techniques may also facilitate recognition of leaks or injuries, greatly expediting the surgical procedure. This visualization may be performed using a conventional endoscope or in some methods a modified cooled CCD or CMOS camera specifically adapted for these procedure. These methods are not limited to the above examples, but rather can be applied to any anatomic tube, duct, lumen, vessel, chamber or hollow structure.

One embodiment of the invention includes the viewing of bioluminescent illumination with a red light background in order that background anatomy with visible light can be viewed at the same time as the bioluminescent image. In terms of human vision, this is optimal if a green signal, generated by the bioluminescent system, and a red signal, generated by a lamp or an LED, are used (Nathans J (1999)). The evolution and physiology of human color vision: insights from molecular genetic studies of visual pigments. Neuron: 299-312. This is accomplished with the aid of a conventional color endoscope camera which has two narrow band interference filters. Endoscopes equipped with interference filters are well known in the prior art for the protection of the surgeon to filter out light from a YAG laser when using this laser for cutting through the endoscope (U.S. Pat. No. 4,916,534, Endoscope, Apr. 10, 1990). Endoscopes with rotating interference filters have been used to remove infrared light from the illuminating lamp (U.S. Pat. No. 5,993,037, Light Source Device for Endoscopes, Nov. 30, 1999). Endoscopes used for fluorescent studies, such as cystoscopes in the bladder, have been equipped with interference filters to isolate the fluorescence signal (U.S. Pat. No. 5,984,861 Endofluorescence Imaging Module For An Endoscope, Nov. 16, 1999).

As to the requisite interference filters, the construction of such filters is well known in the prior art in the visible regions of the electromagnetic spectrum (U.S. Pat. No. 2,890,624, Interference Color Filter With Blue Absorbing Layers Jun. 16, 1959) and infrared (U.S. Pat. No. 4,832,448, Interference Filters May 23, 1989). A dichroic mirror could alternatively be employed in order to reduce the total amount of undesired light admitted in to the system (U.S. Pat. No. 4,047,805, Ripple-Free Dichroic Mirrors, Sep. 13, 1977). Specific bands could also be eliminated if desired (U.S. Pat. No. 5,400,174 Optical Notch Or Minus Filter Mar. 21, 1995) although broadband filters are to be most preferred. The filters could be located at the viewer's end, such as in the glasses of the surgeon (U.S. Pat. No. 6,369,964 Optical Filters For Reducing Eye Strain, During Surgery Apr. 9, 2002) but this is not a highly preferred method because the resolution of the system should be higher if light filtering is done prior to conversion to an electronic signal by the camera. In summary, it is seen that the use of various wide and narrow band interference and dichroic filters is a well known method in the Prior Art to separate a red signal (illuminating the interior of the anatomical structure) from the blue-green signal provided by the bioluminescent composition.

As to the required source of red light, it could be obtained from a conventional high pressure xenon arc lamp by means of conventional interference filters (U.S. Pat. No. 6,364,829 Autofluorescence Imaging System For Endoscopy Apr. 2, 2002) but is probably most conveniently obtained by the use of a light emitting diode. Recent work in this area has provided high-output narrow band devices which do not have temperature sensitivity or wavelength limitations, an issue with some older devices. For example, see U.S. Pat. No. 6,829,271, Light-Emitting Semiconductor Device Producing Red Wavelength Optical Radiation, Dec. 7, 2004; U.S. Pat. No. 7,071,490 Group Iii Nitride Led With Silicon Carbide Substrate Jul. 4, 2006). These devices are small and can readily be incorporated into the endoscopic probe which enters the patient. The advantage which this presents is that the fiber-optical assembly is not needed to carry the incoming visible or infrared light signal. Indeed, in one modification of the endoscope which is useful in the present context both the camera and the red light source could be located on a trocar-like probe which would enter a cavity within an organ of the patient, and no fiber optical whatsoever would be required. An advantage of a conventional fiber optic-based endoscope would be that a very low light level image-intensified, cooled CCD camera could be employed. For example, see U.S. Pat. No. 7,129,464 Low-Photon Flux Image-Intensified Electronic Camera Oct. 31, 2006.

However, our bioluminescent studies in animals in accordance with the present application as described in the specification have provided study results of sufficient brightness that use of a cooled CCD camera has not been necessary, nor has an image intensifier been needed, Indeed, it is a major advantage of the methods and procedures described within the present application that simple commercial video and still cameras of consumer-grade quality are more than sufficient to record the bioluminescent signal, and that generally it is as bright as to be visible with the use of an unmodified, off-the-shelf endoscope. Wavelengths other than the red could be used, provided that the CCD camera had adequate sensitivity in the desired wavelength region. Low light level CCD cameras sensitive in the infrared region are well known in the prior are (for example see U.S. Pat. No. 7,016,518, Vehicle license plate imaging and reading system for day and night, Mar. 21, 2006.) Image intensifiers of Generation III and IV are also commercially available which are extremely sensitive in the near-infrared region of the electromagnetic spectrum.

Examples of experiments using bioluminescence systems that could be implemented using the techniques of the present invention are as follows.

### Example 1

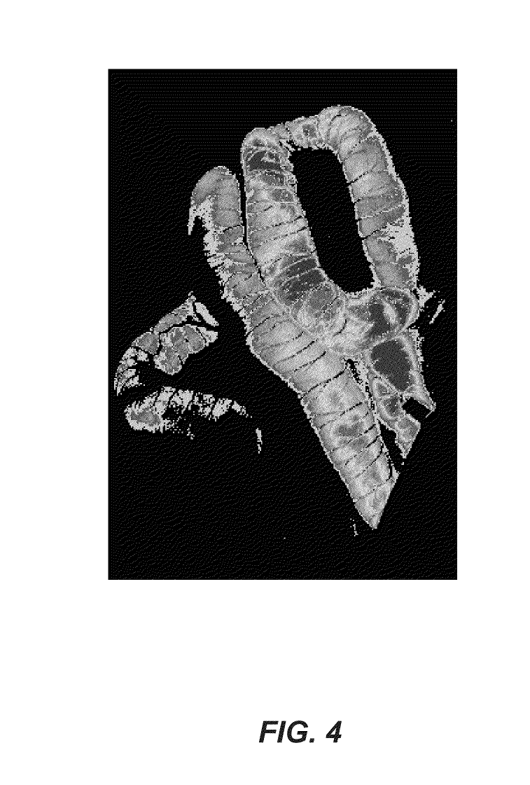

A duodenal loop in the rat can be cannulated and a bioluminescent generating mixture (a solution of coelenterazine (50 mM) in Hank's balanced salt solution and Renilla luciferase, 5 mg/ml in Hank's balanced salt solution) applied thereto. An pseudocolored image with Scion Image according to the light level obtained by a Princeton instruments camera is shown at FIG. 4.

### Example 2

Swine studies. All animal study protocols were approved by the appropriate IACUC, either at the University of Arizona in Tucson, or at the High Quality Research facility located in Ft. Collins, Colo. For these studies, one live pig, approximately 30 kg (anesthetized, non-survival, Arizona site) and one fresh cadaver pig, approximately 30 kg (heparinized, euthanized, Colorado site) were used. A standard open laparotomy operative approach was done to allow use of more than one camera at a time. The color cooled CCD camera used was a Spot R3 supplied by Diagnostic Instruments Inc., Sterling Heights, Mich. This camera is connected by a firewire connection to laptop running Windows XP. The camera is cooled electronically, with its own power source and fan. The camera is controlled and settings adjusted with a Beta Version of the Spot Camera software. Focus is adjusted at the lens for the focal length chosen, between 1 and 2 feet for the open recordings. The videocamera used was a Sony (Sony DCR-SR200) model, which is a hand held camera, mounted on a tripod for the experiments. The image in real time was viewed on the cameras LCD panel or using a small television set. The camera is controlled and settings adjusted on the LCD touch screen. Focus for low light recording is best done in manual mode on the touch screen for the focal length chosen, between 1 and 2 feet for the open recordings.

### Example 2A

Swine bioluminescent cholangiography was done by direct gallbladder puncture and infusion 40 ccs of bioluminescent media using an 18 gauge angiocatheter. Some retraction on the gallbladder in the same method as for a cholecystectomy was done, specifically lifting and moving the gallbladder cephalad to better expose the neck of the gallbladder—cystic duct junctions. The picture was also converted to monochrome to allow comparison to standard radiologic cholangiogram techniques.

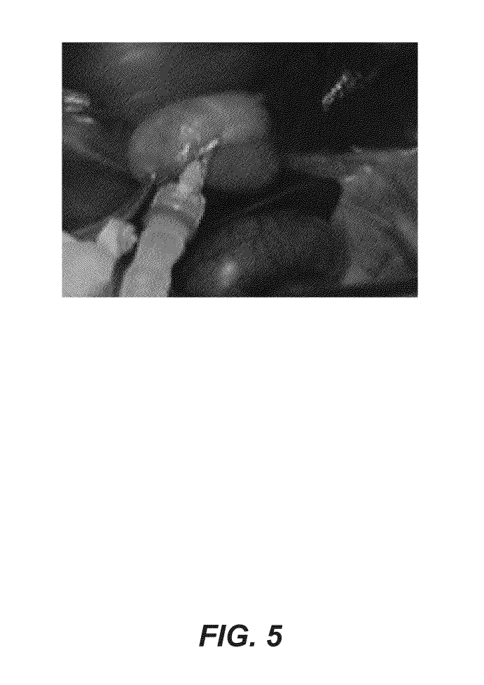

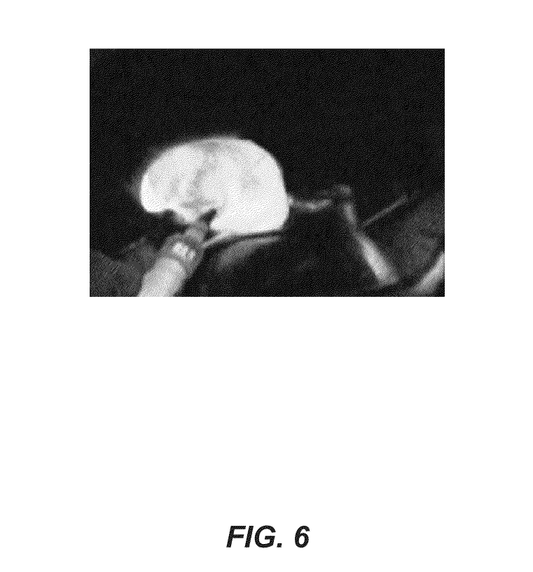

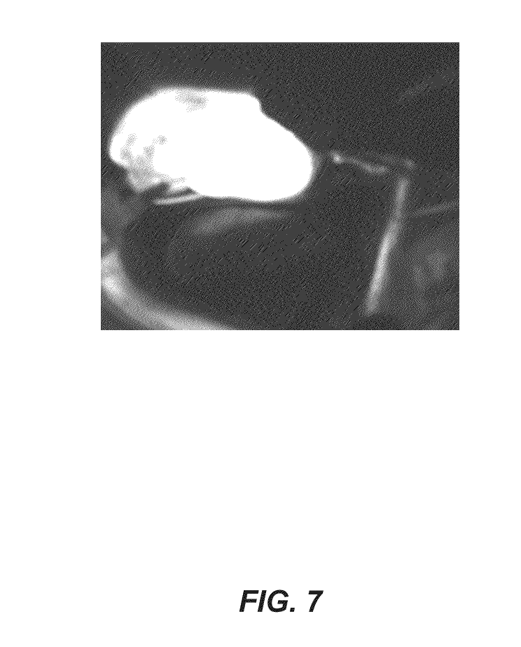

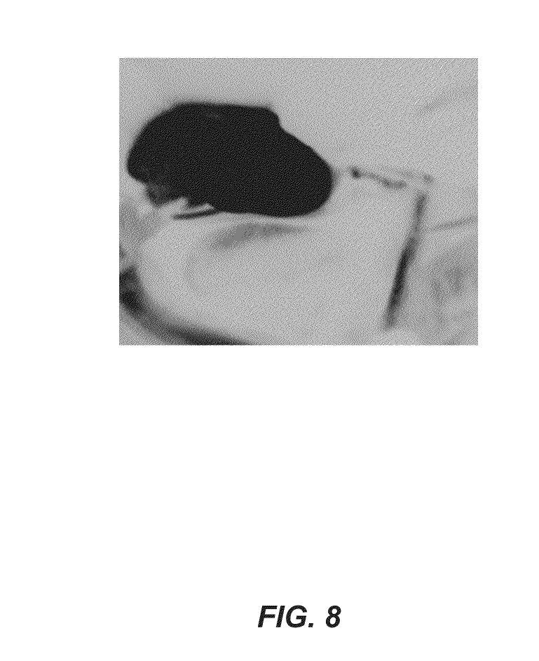

The standard visible light view of a gallbladder (bile ducts not visualized) is shown at FIG. 5. A color bioluminescent cholangiogram is shown at FIG. 6. A monochome bioluminescent cholangiogram is shown at FIG. 7. An inverted monochome bioluminescent cholangiogram is shown at FIG. 8.

### Example 2B

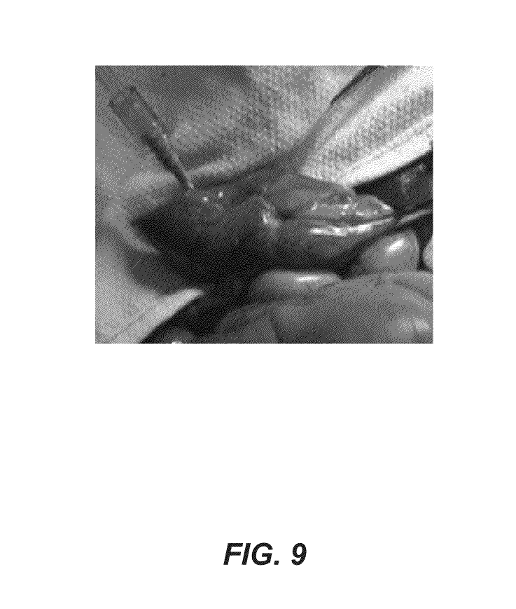

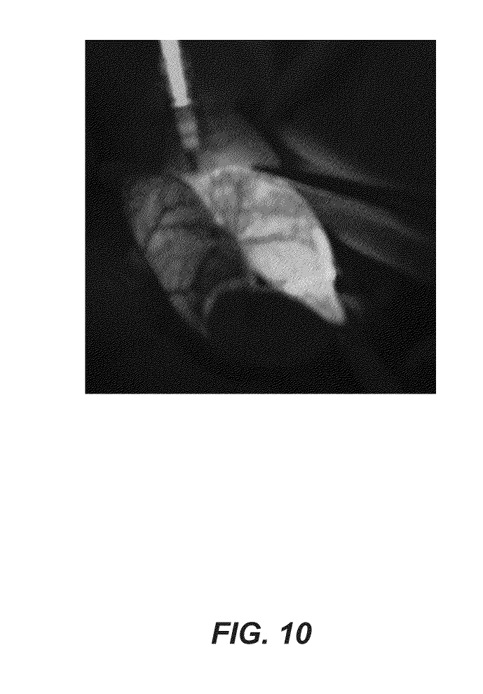

Swine bioluminescent small intestine anastomosis integrity testing was done by direct puncture and infusion of 40 ccs of bioluminescent media using an 18 gauge angiocatheter into the lumen of side to side, stapled small intestine anastomosis. The color cooled CCD camera used was a Spot R3 supplied by Diagnostic Instruments Inc. The standard view of bowel anastomosis is shown at FIG. 9. A color bioluminescent view of bowel anastomosis is shown at FIG. 10.

### Example 2C

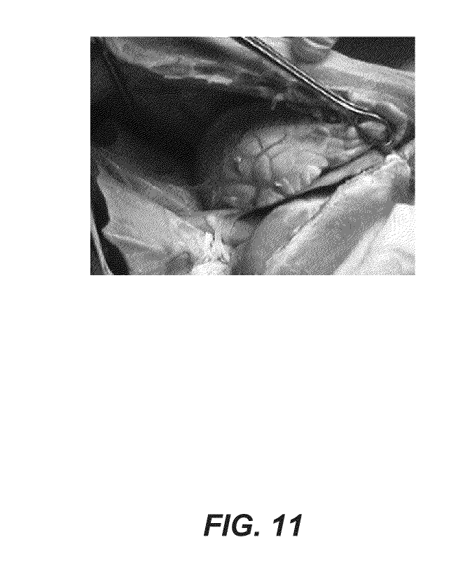

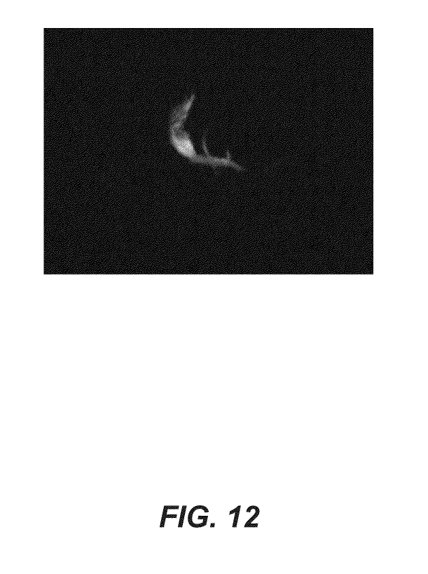

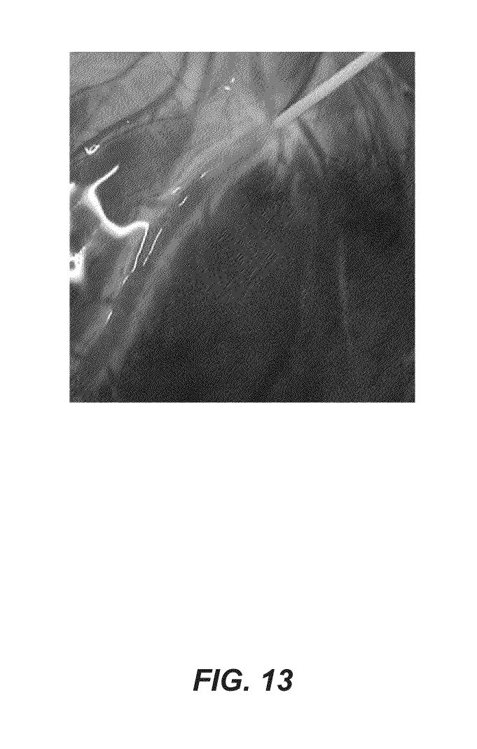

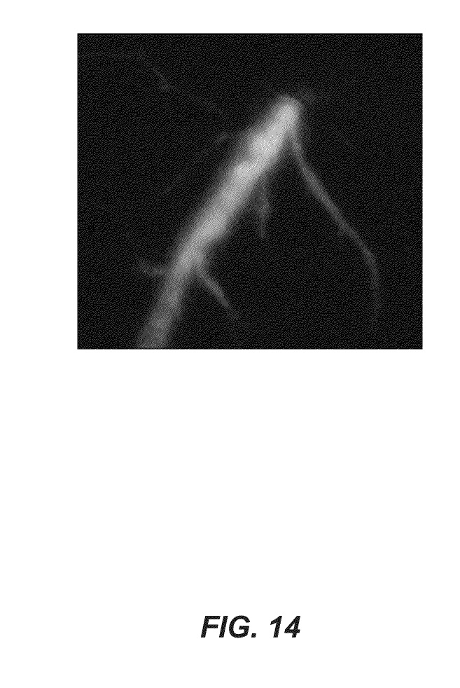

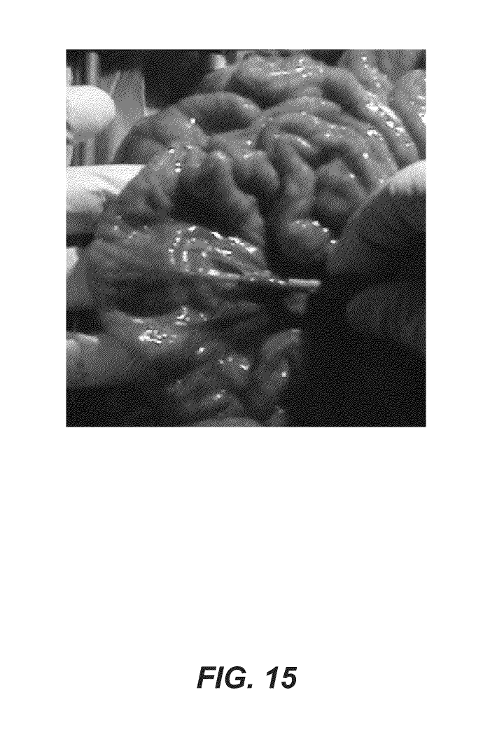

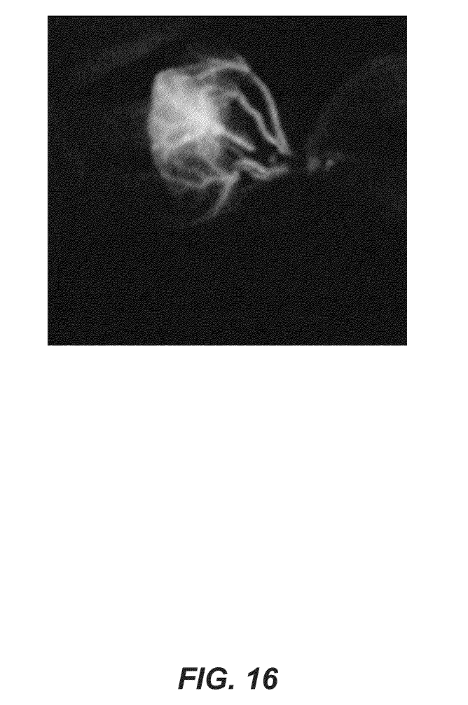

Swine bioluminescent angiography was done was done by direct puncture and infusion of 40 cc of bioluminescent media using an 18 gauge angiocatheter into the pulmonary vasculature, coronary vasculature and small bowel mesentery vasculature, images were from the Sony videocamera. A standard view of the lung right upper lobe is shown at FIG. 11. A color bioluminescent view of the lung right upper lobe is shown at FIG. 12. A standard view of the heart is shown at FIG. 13. A color bioluminescent view of the coronary artery of the heart is shown at FIG. 14. A standard view of the small intestine is shown at FIG. 15. A color bioluminescent view of the mesentery small intestine is shown at FIG. 16.

The foregoing examples establish the value of the present invention in illuminating and highlighting delicate structures.

What is claimed is:

1. A method of using bioluminescence to illuminate internal body structures of a subject comprising:
providing a luciferase solution;
providing a bioluminescence substrate;
combining the luciferase solution and the bioluminescence substrate, and any additionally required activator, ex vivo to initiate bioluminescence in the mixture thereof;
administering the mixture to one or more body structures of a subject; and
viewing the one or more body structures of the subject.

2. The method of claim 1, wherein the step of combining comprises combining the luciferase solution and the bioluminescence substrate in an in-line mixing chamber receiving fluid inputs from two or more fluid conduits and providing a fluid output to at least one fluid conduit.

3. The method of claim 2, wherein the step of combining comprises simultaneous injection of the luciferase solution and the bioluminescence substrate into a Y tube.

4. The method of claim 2, wherein said mixture fluid output is directed into a hollow viscus organ.

5. The method of (Jai 4, wherein said hollow viscus organ comprises a blood vessel, colon, ureter, bladder, bile duct, or cystic duct

6. The method of claim 2, wherein the step of administering comprises
injecting, perfusing, superfusing, infiltrating, or applying the mixture to internal body structures to provide bioluminescent illumination to said body structures.

7. The method of claim 2, wherein the step of viewing comprises obtaining one or more images of said one or more body structures with a camera system sensitive to a wavelength of emitted light of the bioluminescence mixture.

8. The method of claim 7, wherein said one or more images are obtained in dim visible light conditions.

9. The method of claim 7, wherein said one or more images are color or monochrome images.

10. The method of claim 2, wherein the step of viewing comprises surgical viewing.

11. The method of claim 10, wherein the step of viewing comprises endoscopic examination of the one or more body structures.

12. The method of claim 2, wherein the step of viewing further comprises viewing the one or more body structures of the subject with visible light illumination, said visible light comprising visible light in a wavelength range which does not include light in a wavelength range of light emitted by the bioluminescence mixture.

13. The method claim 12, wherein the visible light illumination is a red light illumination.

14. The method of claim 9, further comprising: comparing the one or more body structures as illuminated by the bioluminescence mixture to the one or more body structures illuminated with visible light illumination.

15. A method of examining an animal, avian or human object, comprising the steps of:
providing a luciferase solution;
providing a bioluminescence substrate;
combining the luciferase solution and the bioluminescence substrate, and any additionally required activator, ex vivo to initiate bioluminescence in the mixture thereof;
administering the mixture to one or more body structures of a subject; and
viewing the one or more body structures of the subject.
obtaining one or images of said one or more body structures with bioluminescent illumination with a camera system sensitive to a wavelength of emitted light of the bioluminescence mixture.

16. The method of claim 15, wherein the step of combining comprises combining the luciferase solution and the bioluminescence substrate in an in-line mixing chamber receiving fluid inputs from two or more fluid conduits and providing a fluid output to at least one fluid conduit.

17. The method of claim 16, wherein the step of combining comprises simultaneous injection of the luciferase solution and the bioluminescence substrate into a Y tube.

18. The method of claim 17, wherein said mixture is injected into a hollow viscus organ.

19. The method of claim 18, wherein said hollow viscus organ comprises a blood vessel, colon, ureter, bladder, be duct, or cystic duct.

20. The method of claim 19, wherein said one or more images are obtained in dim visible light conditions.

21. The method of claim 19, wherein said one or more images are color or monochrome images.

22. The method of claim 19, further comprising obtaining one or more images of the one or more body structures of the subject with visible light illumination, said visible light comprising visible light in a wavelength range which does not include light in a wavelength range of light emitted by the bioluminescence mixture.

23. The method of claim 22, wherein said images are obtained with a camera system sensitive to a wavelength of the visible light illumination.

24. The method claim 23, wherein the visible light illumination is a red light illumination.

25. The method of claim 23, further comprising: comparing said image with bioluminescent illumination with said image with visible light illumination.

26. The method of claim 25, further comprising a step of switching between said image with bioluminescent illumination and said image with visible light illumination.

27. The method of claim 22, wherein said images are obtained by an endoscopic examination of the object.
